(19) United States
(12) Patent Application Publication
(10) Pub. No.: US 2016/0271340 A1
(43) Pub. Date: Sep. 22, 2016

(54) RETRACTABLE SYRINGE

(71) Applicant: GLOBAL MEDISAFE HOLDINGS LIMITED, Fullerton Cove (AU)

(72) Inventor: Allan WALSH, New South Wales (AU)

(21) Appl. No.: 15/031,930

(22) PCT Filed: Dec. 17, 2013
(86) PCT No.: PCT/IB2013/061006

§ 371 Date: Apr. 25, 2016

(30) Foreign Application Priority Data
Oct. 25, 2013 (AU) 2013904118

(51) Int. Cl. A61M 5/32; A61M 5/315

(52) U.S. Cl. CPC A61M 5/3221; A61M 5/31511

(57) ABSTRACT

An auto-retractable syringe having a barrel and a plunger moveable within said barrel, said barrel having a gland assembly inside a forepart thereof, said gland assembly being connected to a needle hub; wherein said needle hub is located in the forepart of said barrel via a Luer-lock thread, internal of the forepart of the barrel, and two or more lugs protruding from said needle hub, and wherein said Luer-lock thread is open internal of the barrel such that the needle hub can be drawn into the barrel uninhibited by the thread.

### TECHNICAL FIELD

The invention relates to the field of hypodermic syringe manufacture. In particular, the invention relates to a retractable safety syringe, in which the needle is capable of being withdrawn into the barrel of the syringe after use via a spring or similar mechanism.

### BACKGROUND OF THE INVENTION

Safety syringes for hypodermic needles are becoming a very important tool of healthcare. The ability to prevent needle-stick injuries is extremely important in preventing the spread of blood-borne diseases.

One particular type of safety syringe is the retractable syringe. That is: a syringe where there is provided a mechanism, usually a spring, inside the syringe that is activated upon completion of the injection of the fluid, whereby the spring acts to retract the needle inside the syringe barrel. This prevents the needle from causing a needle-stick injury, and in particular it allows the needle operator to withdraw the needle via a one-handed operation, which makes it much easier to achieve in the situation of providing injections to patients in a busy hospital or clinic.

One drawback with some existing retractable syringes is that they tend to withdraw quit rapidly and without allowing the operator to control the speed of retraction. This can cause the operator or patient to flinch which is not desirable.

Other designs are relatively complex and so are difficult or expensive to manufacture. Yet other designs do not allow for full dispensing of the injectable fluid, due to dead-zones being left in the barrel.

Accordingly, it is an object of the invention to provide a retractable syringe construction that ameliorates at least some of the problems associated with the prior art.

### SUMMARY OF THE INVENTION

According to a first aspect of the invention, there is provided an auto-retractable syringe having a barrel and a plunger moveable within said barrel, said barrel having a gland assembly inside a forepart thereof, said gland assembly being connected to a needle hub; wherein said needle hub is located in the forepart of said barrel via the interaction of a Luer-lock thread, internal of the forepart of the barrel, and two or more lugs protruding from said needle hub, and wherein the end of said Luer-lock thread is open internal of the barrel such that the needle hub can be drawn into the barrel uninhibited by the thread.

This invention also allows a more controllable retraction of the needle within the barrel. The base of the needle hub rests on the fore-surface of the gland assembly and after being located in place by the Luer-thread. After injection is completed, a protrusion at the end of the plunger may pass through the gland and to make a locked connection with the fore-end of the gland, allowing the protrusion to be used to pull the gland, and thereby the needle hub, back into the barrel.

The fact that the Luer-lock passage is open at the internal end allows the needle to be retracted easily and in a controlled manner that avoids such problems a blood spatter, which can occur with the too-rapid retraction that is known in the prior art.

Preferably, the barrel is of a two-part construction, and an internal recess is defined by a discontinuity of the inside surface of the barrel, such as an internal notch, which may be formed at the junction of the two barrel parts.

Preferably, the Luer-lock thread is formed integrally with the barrel, and a spring is disposed in between the barrel and the gland assembly and engages both such that the spring tends to urge the needle hub into the barrel and away from the forepart of the barrel. Such a syringe has a relatively simple construction, which has advantages for mass production and reliability of operation.

Preferably, the forepart of the plunger has a barbed protrusion that is adapted to reach through the gland assembly hub and engage with the forepart of same such that said protrusion cannot readily be withdrawn through the gland assembly.

It is a particularly advantageous embodiment wherein the gland assembly is, prior to use, prevented from withdrawal into the barrel by two or more flexible barbs protruding outwardly from the gland assembly and engaging with a recess in the inner wall of the barrel. Further advantageously, said barbs may be caused to disengage with said notches by the action of the plunger engaging with the gland assembly, thereby allowing retraction of the gland assembly into the barrel.

The above construction is preferred as it is a relatively simple and efficient way to release the gland assembly from its operating position and allowing the spring to draw the needle into the barrel, as it acts to push the gland assembly into the barrel.

A preferred construction of the above is one wherein said flexible barbs protrude from the rearward end of the gland assembly; and wherein the forepart of the plunger is adapted to operatively engage with the gland assembly and thereby cause the barbs to disengage with said notches as the plunger is forced against the gland assembly.

This embodiment is particularly advantageous, as it enables the plunger to close all space in the between its fore-end and the gland assembly, promoting full dispensing of the injectable fluid.

It is particularly preferred that the barbs branch off the gland assembly, thereby forming a v-shaped gap between the gland assembly and the barb; and wherein the engagement of the plunger with the barb causes said gap to close completely.

In addition to the advantages described above, the invention improves the ability of the syringe operator to render the syringe disabled from further use via a one-handed operation.

This advantage is further enhanced when the invention provides, in a further aspect, a syringe according to that described above, further incorporating a plunger that includes: a shaft that is formed having a four-vaned cross profile, collocated notches on each vane that define a preferred breaking zone, and located on two of said vanes, nearer to a forepart of said shaft than said notches, two resilient barbs extending outward from the vanes; said barbs being adapted to not interfere with the withdrawal of the shaft from the barrel, but to prevent the shaft being pushed back into the barrel.

This arrangement allows the shaft to be broken by, for example, thumb pressure on the area above the notches, and the barbs prevent the remaining end of the shaft from being forced into the barrel and potentially causing the needle to be forced back out of the fore-end of the barrel.

A particularly advantageous embodiment of this aspect of the invention is provided wherein said barbs are formed in a branched arrangement with the vanes such that a gap is present between the barb and the vane.

Preferably, the rearward end of the barrel has slots adapted to receive the vanes of the shaft, and each said slot internally features a wedge-shaped stop that has an angled face toward the forepart of the plunger, and a perpendicular surface facing toward the rearward end of the plunger that engages the barbs and blocks their path back into the barrel.

Now will be described, by way of a specific, non-limiting example, a preferred embodiment of the invention with reference to the drawings.

### BRIEF DESCRIPTION OF THE DRAWINGS

FIG. 1 is a cross-sectional view of a retractable syringe according to the invention.

FIG. 2 shows a number of views of a barrel fore-section as per the syringe of FIG. 1.

FIG. 3 shows a number of views of a barrel rear-section as per the syringe of FIG. 1.

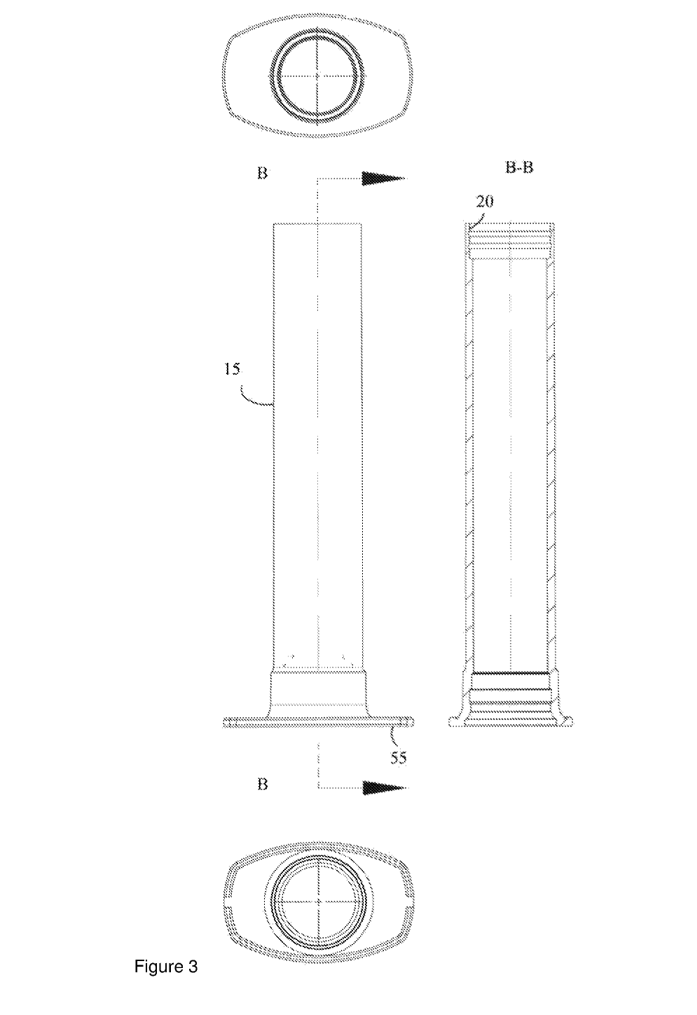

FIG. 4 shows a number of views of a gland assembly as per the syringe of FIG. 1.

FIG. 5 shows a number of views of a plunger as per the syringe of FIG. 1.

FIG. 6 shows a number of views of a piston to be positioned on the fore-end of a plunger, as per the syringe of FIG. 1.

FIG. 7 shows a number of views of a guide to be positioned inside the rearward end of a syringe barrel, as per the syringe of FIG. 1.

FIG. 8 shows the syringe of FIG. 1 in operation, nearing the conclusion of an injection sequence.

FIG. 9 shows the syringe of FIG. 1 in operation, nearing the conclusion of a needle retraction sequence.

FIG. 10 shows the syringe of FIG. 1 in operation, at the conclusion of a needle retraction sequence.

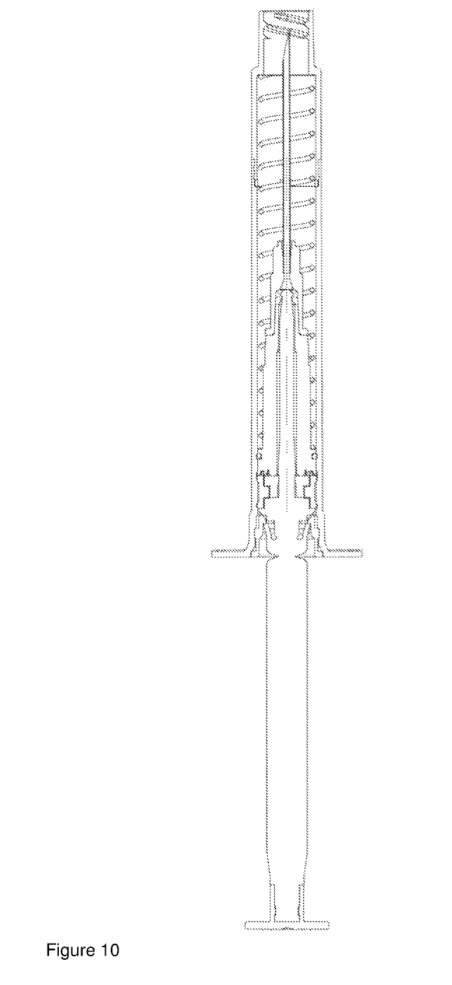

### DETAILED DESCRIPTION OF THE INVENTION

The figures show a syringe according to the invention, both in an assembled and in a disassembled state, to illustrate the salient features of each part and their working interactions.

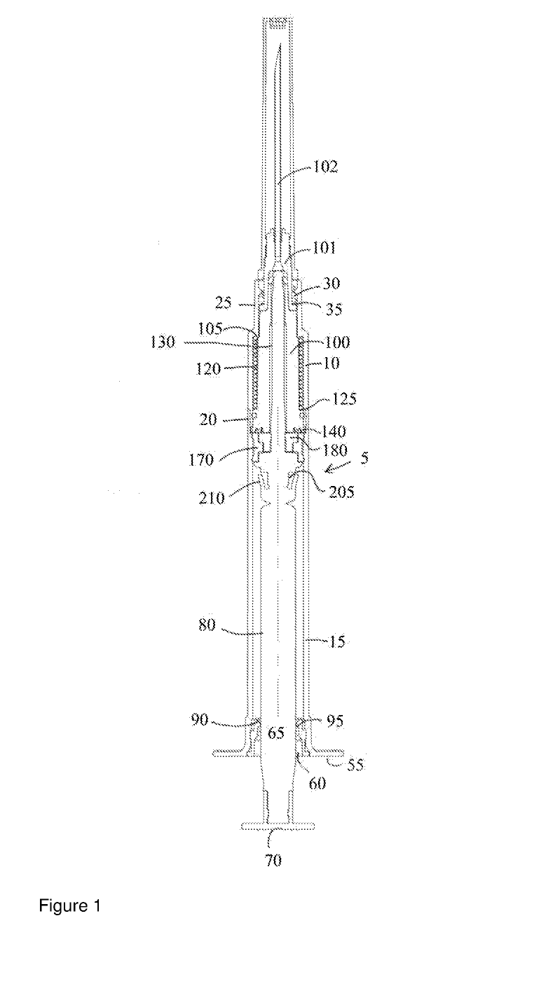

FIG. 1 shows the main features of a barrel of a retractable syringe according to the invention. The barrel 5 is formed by two tubes: the fore tube 10 and the rear tube 15, both having equal internal diameter. The fore tube 10 fits inside a widening 20 of the rear tube 10.

The fore-tube 10 at its fore-end 25 is narrower and has an internal Luer-lock thread 30. Importantly, the threaded section 25 is completely open to the internal of the rest of the tube 10, such that a compatible part that reaches the internal end 35 of the thread 30 could pass directly into the tube, unhindered.

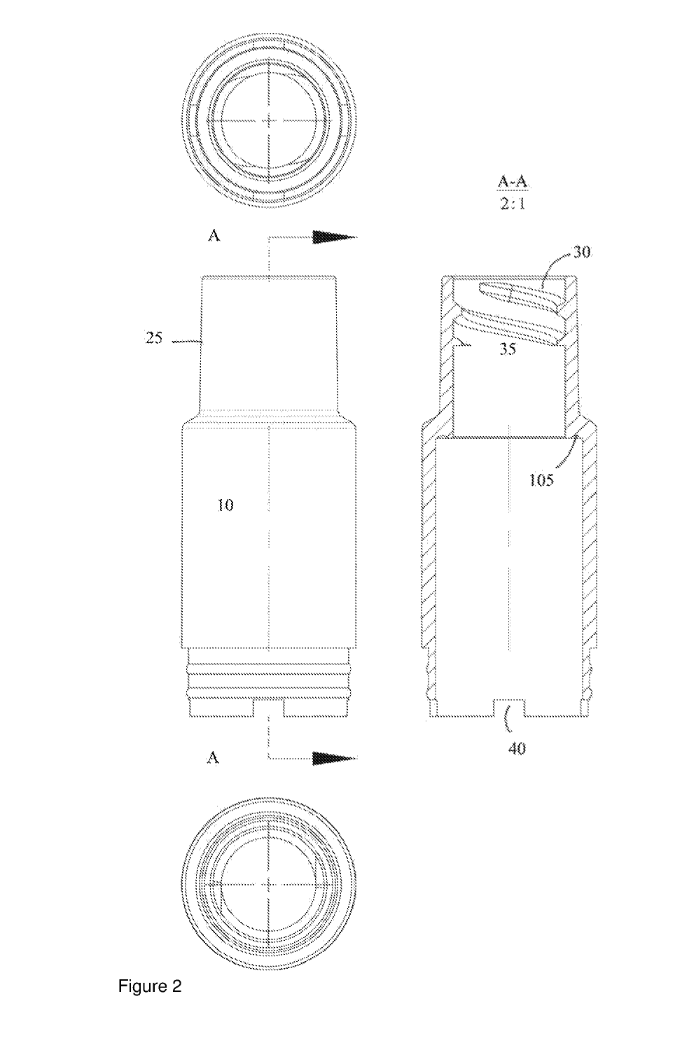

At the junction of the two tubes (10, 15) are formed internal notches 40, which are discontinuities of the internal surface of the barrel 5. These are defined by a small section of the wall of the fore-tube 10 being removed. These features of the fore-tube 10 are illustrated in greater detail in FIG. 2.

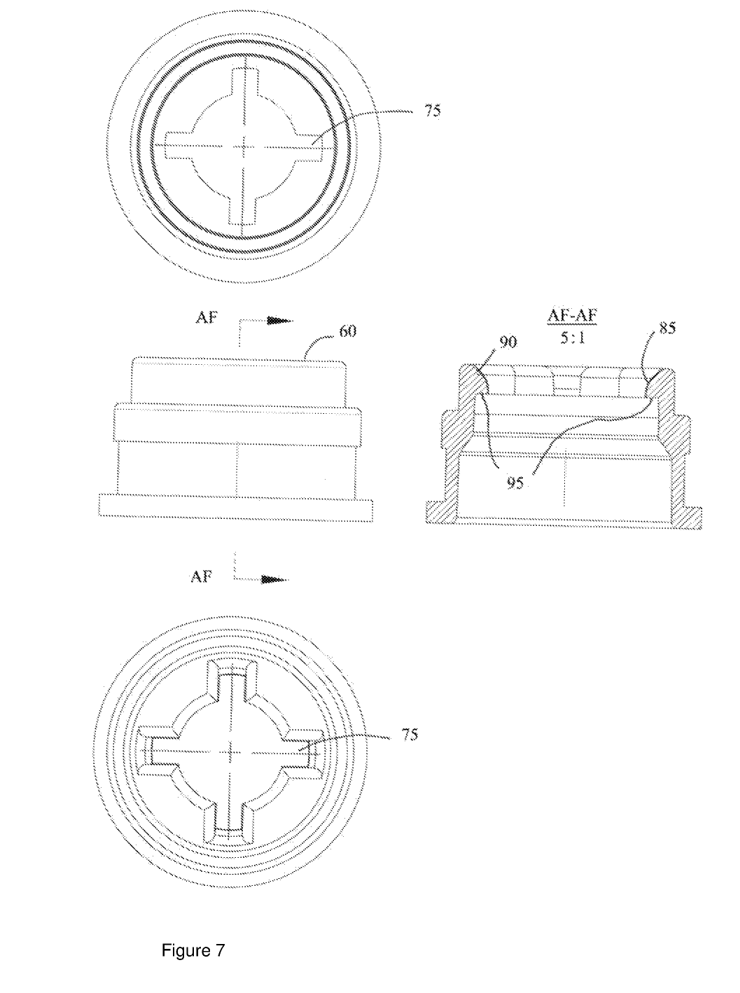

The rear end of the barrel is flanged 55 and has an aperture defined by an insert 60 that is adapted to allow the shaft 65 of the plunger 70 to pass through. As can be seen in FIG. 7, the aperture 60 is a circular hole with four arm-slots 75 protruding therefrom, that accommodate the vanes 80 of the plunger 70. Inside each slot 75 there are wedge-shaped constrictions 85 that have angled surfaces 90 facing toward the fore-end of the barrel 5 and perpendicular surfaces 95 facing toward the rear of the barrel 5.

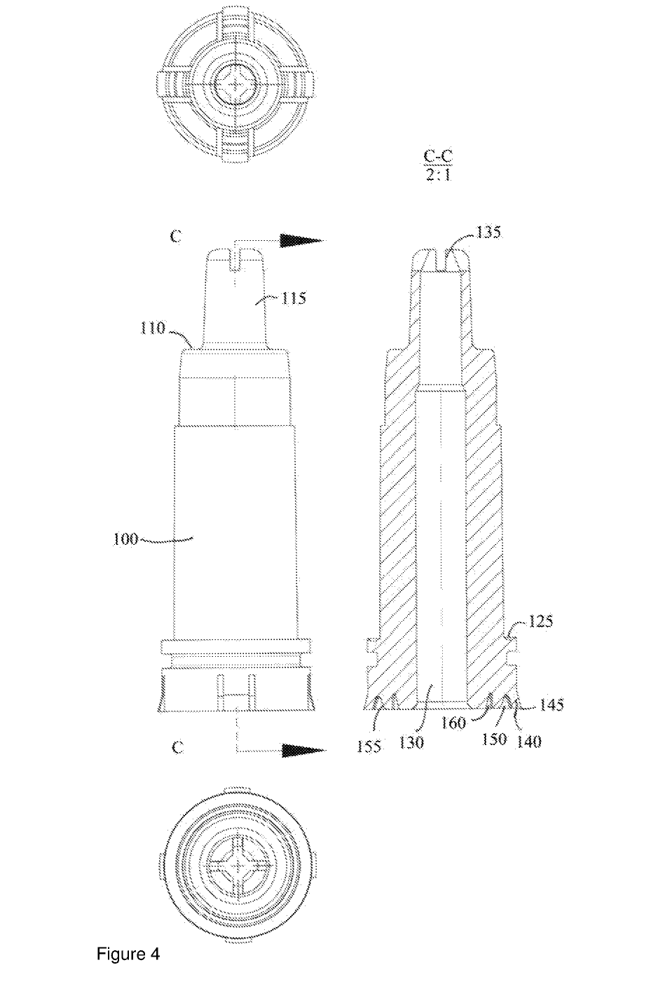

The gland assembly 100 shown in FIG. 4 fits inside the barrel 5 at is fore-end. It abuts the rear end of the needle hub 101 via its front shoulder 110. The nose-section 115 protrudes into the threaded section 25, inside the needle hub 101. A spring 120 is located around the outside of the gland. It is adapted to make contact with: an internal rearward facing shoulder of the barrel 105; and with a flange 125 on the surface of the gland 100, tending to force the two parts apart, unless otherwise constrained.

The gland 100 is hollow, having a passage 130 through its centre. At the fore-end of the passage, there is a resilient constriction 135.

At the rear end of the gland 100, and illustrated in FIG. 4, there are four flexibly branched protrusions 140. The outer point of said protrusions feature a wedge-shaped barb 145. There is also an inner notch 150 with an inwardly sloped surface 155. There is a gap 160 formed between the protrusions 140 and the gland 100.

When the syringe is assembled, the four barbs 145 are located in the notches 40 on the inner surface of the barrel 5. These barbs 145 prevent the gland 100 from being moved toward the rear of the barrel 5 by the action of the spring 120.

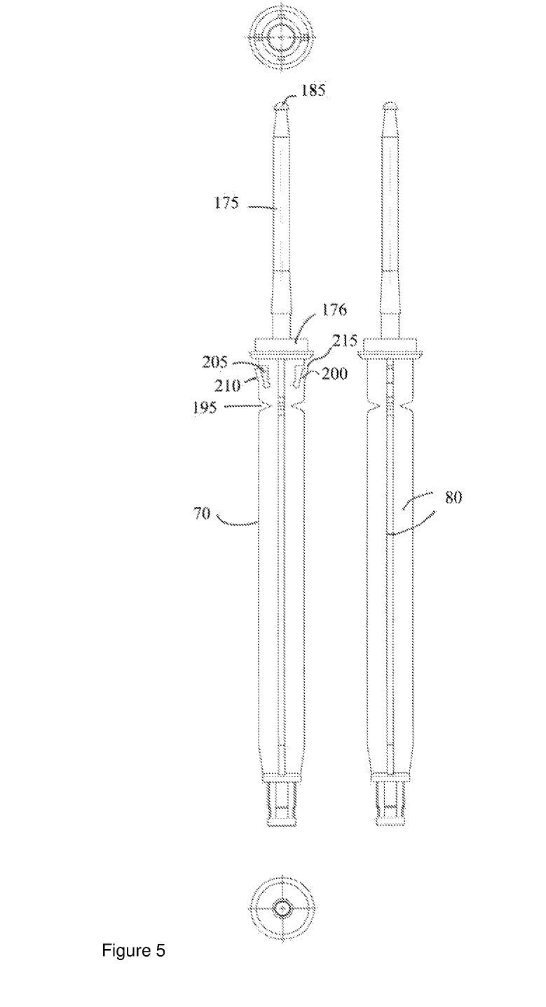

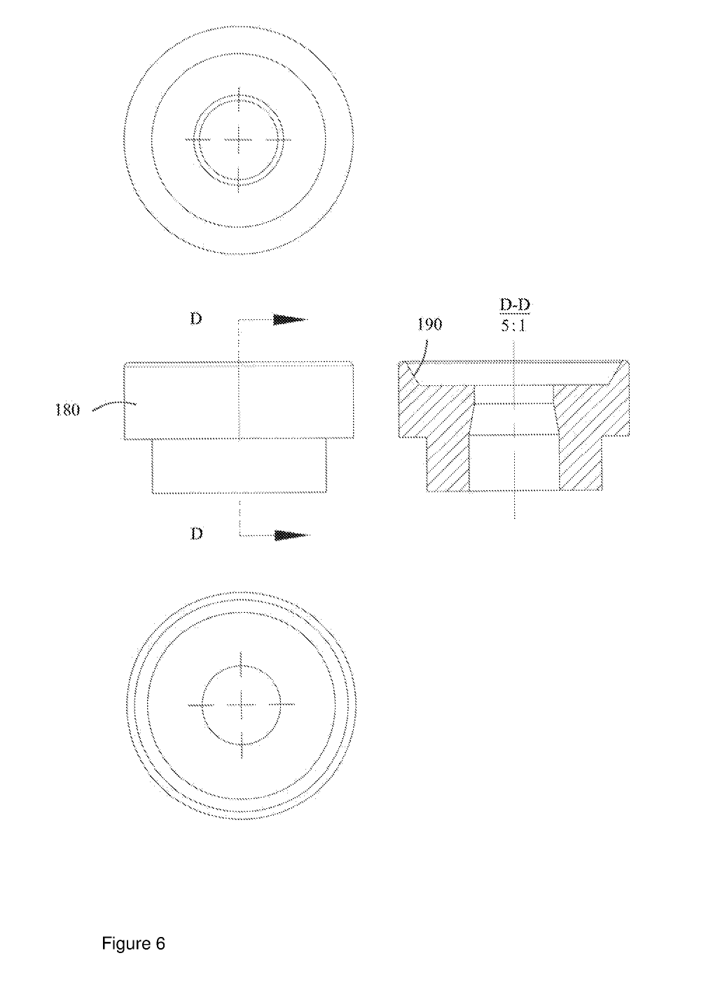

As illustrated in FIGS. 1, 5 and 6, the syringe also incorporates a plunger 70. The plunger has a shaft 65 that is defined by four perpendicular vanes 80. Illustrated in FIG. 1 is a piston that is adapted to be fitted on to the plunger 70 at a platform 176.

At the fore-end of the plunger 70 is a barbed protrusion 175 and a sealing ring 180. The protrusion 175 has a barbed end 185. The sealing ring 180 has an inwardly sloping surface 190 facing the fore-end of the plunger.

The vanes 80 of the plunger 70 each have collocated v-shaped notches 195 that define a preferred breaking point. Located between said notches 195 and said piston 170 on only two opposite vanes 80 are two resilient barbs 200. These barbs 200 are branched off the vanes 80, forming a v-shaped gap 205. The barbs 200 have an angled surface 210 that faces the rearward end of the plunger 70, and a perpendicular surface 215 facing toward the fore-end of the plunger 70.

In operation, the syringe is filled via a filling needle, which is attached to the syringe as per the injecting needle hub 101 in FIG. 1. The injection fluid is then drawn into the barrel of the syringe in the usual manner by manual withdrawal of the plunger. The filling needle is then removed and an injecting needle 102 is then screwed in via the Luer-lock thread until it abuts the fore-end of the gland 100. The needle hub 101 is adapted to fit over the nose-end 115 of the gland 100.

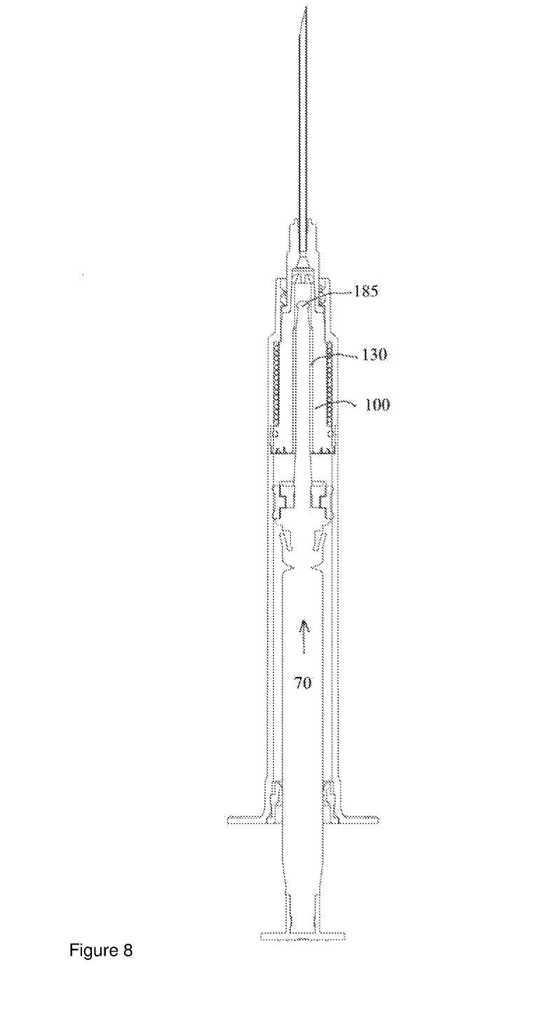

As illustrated in FIG. 8, the plunger 70 is used to dispense the fluid in the usual manner. Once dispensing is complete, the barbed protrusion 185 on the plunger has travelled through the hollow passage 130 in the gland 100, thereby pushing all fluid out of the syringe.

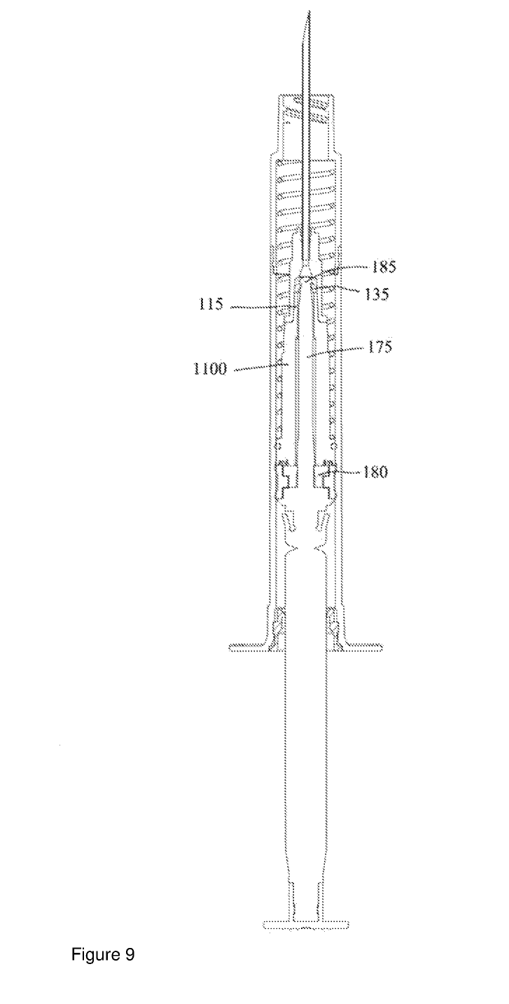

As shown in FIG. 9, the barbed end 185 of the protrusion 175 makes a locking engagement with the constricted part 135 of the nose-end 115 of the gland 100.

At the same time, the sealing ring 180 is forced against the rear end of the gland assembly 100. The sloped surface 190 of the sealing ring 180 contacts the sloped surface 155 of the protrusions 140 and the two surfaces co-operate to pull the protrusions 140 inward. This closes the gap 160 and brings the barbs 145 inward sufficiently to disengage from the notches 40 and the inner surface of the barrel 5.

As the barbed protrusion 175 has now locked the plunger 70 into the gland, they now cannot be separated. As the barbs 145 have been withdrawn, the gland 100 with needle hub 101 attached can now be pulled back into the barrel 5 under the power of the spring 120, with the speed of retracting being governed by thumb pressure on the end 65 of the plunger 70. Because the needle hub is clear of the Luer-thread 25 via the gap 35, it is not hindered from retraction.

As the barbs 210 on the shaft vanes 80 pass through the hole 60, they are temporarily forced inward by the co-operation of the sloped surfaces 210 and 90, and once the barbs 210 are clear of the wedges 85, the plunger 65 is prevented from returning into the barrel 5 by the locking together of the perpendicular surfaces 215 and 95.

The needle operator can then use e.g. their thumb to snap off the end of the plunger shaft via the break point created by the notches 195.

Thus the syringe is rendered safe, as the needle is withdrawn entirely into the barrel, and there is no easy way to force the needle outward again.

It will be appreciated by those skilled in the art that the above described embodiment is merely one example of how the inventive concept can be implemented. It will be understood that other embodiments may be conceived that, while differing in their detail, nevertheless fall within the same inventive concept and represent the same invention.

For example, an alternative embodiment may be conceived wherein a spring is not included, and the operator may simply manually withdraw the needle hub after injection is complete.

1. An auto-retractable syringe having a barrel and a plunger moveable within said barrel, said barrel having a gland assembly inside a forepart thereof, said gland assembly being connected to a needle hub; wherein said needle hub is located in the forepart of said barrel via a Luer-lock thread, internal of the forepart of the barrel, and two or more lugs protruding from said needle hub, and wherein said Luer-lock thread is open internal of the barrel such that the needle hub can be drawn into the barrel uninhibited by the thread.

2. The syringe of claim 1, wherein the barrel is of a two-part construction.

3. The syringe of claim 1, wherein the Luer-lock thread is formed integrally with the barrel.

4. The syringe of claim 3, wherein the forepart of the plunger has a barbed protrusion that is adapted to reach through the gland assembly hub and engage with the forepart of same such that said protrusion cannot readily be withdrawn through the gland assembly.

5. The syringe of claim 4, wherein the gland assembly is, prior to use, prevented from withdrawal into the barrel by two or more flexible barbs protruding outwardly from the gland assembly and engaging with a recess in the inner wall of the barrel.

6. The syringe of claim 5, wherein said barbs may be caused to disengage with said notches by the action of the plunger engaging with the gland assembly, thereby allowing retraction of the gland assembly into the barrel.

7. The syringe of claim 6, wherein said flexible barbs protrude from the rearward end of the gland assembly; and wherein the forepart of the plunger is adapted to operatively engage with the gland assembly and thereby cause the barbs to disengage with said notches as the plunger is forced against the gland assembly.

8. The syringe of claim 7, wherein the barbs branch off the gland assembly, thereby forming a v-shaped gap between the gland assembly and the barb; and wherein the engagement of the plunger with the barb causes said gap to close completely.

9. The syringe of claim 5, wherein the recess is defined by a discontinuity of the inside surface of the barrel at the junction of the two barrel parts.

10. The syringe of claim 1, wherein a spring is disposed in between the barrel and the gland assembly and engages both such that the spring tends to urge the needle hub into the barrel and away from the forepart of the barrel.

11. The syringe of claim 1, further incorporating a plunger that includes: a shaft that is formed having a four-vaned cross profile, collocated notches on each vane that define a preferred breaking zone, and located on two of said vanes, nearer to a forepart of said shaft than said notches, two resilient barbs extending outward from the vanes; said barbs being adapted to not interfere with the withdrawal of the shaft from the barrel, and adapted to prevent movement of shaft back into the barrel.

12. The syringe of claim 11, wherein said barbs are formed in a branched arrangement with the vanes such that a gap is present between the barb and the vane.

13. The syringe of claim 11, wherein the rearward end of the barrel has slots adapted to receive the vanes of the shaft, and each said slot internally features a wedge-shaped stop that has an angled face toward the forepart of the plunger, and a perpendicular surface facing toward the rearward end of the plunger that engages the barbs thereby preventing them from travelling back into the barrel.
